(19) United States
(12) Patent Application Publication
(10) Pub. No.: US 2026/0256460 A1
(43) Pub. Date: Sep. 3, 2026

(54) ULTRASOUND IMAGING SYSTEM AND AN IMAGING METHOD FOR ULTRASOUND IMAGING SYSTEM

(71) Applicant: GE Precision Healthcare LLC, Waukesha, WI (US)

(72) Inventors: Liye Pei, Wuxi (CN); Jieying Xu, Wuxi (CN); Yimeng Lin, Wuxi (CN)

(21) Appl. No.: 19/554,272

(22) Filed: Mar. 2, 2026

(30) Foreign Application Priority Data
Feb. 28, 2025 (CN) 202510240498.7

(51) Int. Cl. A61B 8/00; G06V 10/10; G06V 10/12; G06V 10/25

(52) U.S. Cl. CPC A61B 8/54; A61B 8/4461; A61B 8/463; A61B 8/5246; G06V 10/12; G06V 10/16; G06V 10/25; G06V 2201/03

(57) ABSTRACT

An ultrasound imaging system including: an ultrasonic transducer, moving between a first scanning position and a second scanning position to acquire an ultrasound image of an object being scanned; and a controller, configured to: detect a region of interest in the ultrasound image in real time during a process in which the ultrasonic transducer moves from the first scanning position to the second scanning position; and dynamically adjust a scanning mode of the ultrasonic transducer according to a detection result of the region of interest, where in different scanning modes, moving speeds of the ultrasonic transducer are different.

### CROSS-REFERENCE TO RELATED APPLICATION

This application claims priority to Chinese Patent Application No. 202510240498.7, which was file on February 28, 2025 at the Chinese Patent Office. The entire contents of the above-listed application are incorporated by reference herein in their entirety.

### TECHNICAL FIELD

Embodiments of the present application relate to the technical field of ultrasound imaging, and in particular, to an ultrasound imaging system and an imaging method for an ultrasound imaging system.

### BACKGROUND

Ultrasound imaging technology is a real-time, non-destructive imaging technology that uses an ultrasonic transducer to send an ultrasonic signal to an object being scanned and receive an ultrasonic echo signal from the object being scanned, and processes the received ultrasonic echo signal to perform imaging.

The image quality and scanning time of ultrasound imaging are important indicators for measuring the performance of an ultrasound imaging system. For example, a high-quality ultrasound image helps a doctor observe tissues and organs more accurately, which is conducive to disease diagnosis. Short scanning time can improve the patient experience, reduce waiting time, improve examination efficiency, and help optimize a device utilization rate and medical resource allocation.

### SUMMARY OF THE INVENTION

Embodiments of the present application provide an ultrasound imaging system and an imaging method for an ultrasound imaging system.

According to an aspect of the embodiments of the present application, an ultrasound imaging system is provided, comprising: an ultrasonic transducer, moving between a first scanning position and a second scanning position to acquire an ultrasound image of an object being scanned; and a controller, configured to: detect a region of interest in the ultrasound image in real time during a process in which the ultrasonic transducer moves from the first scanning position to the second scanning position; and dynamically adjust a scanning mode of the ultrasonic transducer according to a detection result of the region of interest, wherein in different scanning modes, moving speeds of the ultrasonic transducer are different.

According to another aspect of the embodiments of the present application, an imaging method for an ultrasound imaging system is provided, the ultrasound imaging system comprising an ultrasonic transducer, and the method comprising: causing the ultrasonic transducer to move between a first scanning position and a second scanning position to acquire an ultrasound image of an object being scanned, wherein a region of interest in the ultrasound image is detected in real time during a process in which the ultrasonic transducer moves from the first scanning position to the second scanning position, a scanning mode of the ultrasonic transducer is dynamically adjusted according to a detection result of the region of interest, and in different scanning modes, moving speeds of the ultrasonic transducer are different.

With reference to the following description and drawings, specific implementations of the embodiments of the present application are disclosed in detail, and the way in which the principles of the embodiments of the present application can be employed are illustrated. It should be understood that the implementations of the present application are not limited in scope thereby. Within the scope of the spirit and clauses of the appended claims, the implementations of the present application comprise many changes, modifications, and equivalents.

### BRIEF DESCRIPTION OF THE DRAWINGS

The included drawings are used to provide further understanding of the embodiments of the present application, which constitute a part of the description and are used to illustrate the implementations of the present application and explain the principles of the present application together with textual description. Evidently, the drawings in the following description are merely some embodiments of the present application, and those of ordinary skill in the art may obtain other implementations according to the drawings without involving inventive effort. In the drawings:

FIG. 1 is a schematic diagram of an ultrasound imaging system according to an embodiment of the present application;

FIG. 2 is a schematic diagram of the structure of an ultrasound imaging system according to an embodiment of the present application;

FIG. 3 is a schematic diagram of an ultrasound imaging assembly according to an embodiment of the present application;

FIG. 4 is a schematic diagram of internal signal interaction of an ultrasound imaging system according to an embodiment of the present application;

FIG. 5 is a schematic diagram of a control method executed by a controller according to an embodiment of the present application;

FIG. 6 is a schematic diagram of positions of an ultrasonic transducer according to an embodiment of the present application;

FIG. 7 is another schematic diagram of positions of an ultrasonic transducer according to an embodiment of the present application;

FIG. 8 is another schematic diagram of positions of an ultrasonic transducer according to an embodiment of the present application; and

FIG. 9 is a schematic diagram of reconstructed ultrasound images and marking information according to an embodiment of the present application.

### DETAILED DESCRIPTION

Moving speed of an ultrasonic transducer is related to both image quality and scanning time. In a current ultrasound imaging system, in one scanning cycle, the moving speed of the ultrasonic transducer is generally set to a low constant value to obtain good image quality. However, a low constant moving speed leads to an increase in scanning time, and consequently, cannot ensure scanning efficiency of the ultrasound imaging system. A breast ultrasound imaging system is taken as an example. Low-speed movement of the ultrasonic transducer can ensure a better imaging parameter, such as frame density, but leads to low scanning efficiency. Because a travel distance of the ultrasonic transducer is generally fixed in one scanning cycle, slow movement prolongs the scanning time.

One of the beneficial effects of some embodiments of the present application is that: A region of interest in an acquired ultrasound image is detected in real time during a process in which an ultrasonic transducer moves from a first scanning position to a second scanning position; and a scanning mode of the ultrasonic transducer is dynamically adjusted according to a detection result of the region of interest, wherein in different scanning modes, moving speeds of the ultrasonic transducer are different. Therefore, the moving speed of the ultrasonic transducer can be dynamically adjusted based on the real-time detection result of the region of interest, so that a balance between image quality and scanning time can be achieved, which is conducive to shortening the scanning time of ultrasound imaging and improving the scanning efficiency of the ultrasound imaging system while ensuring the image quality of ultrasound imaging.

The aforementioned and other features of the embodiments of the present application will become apparent from the following description with reference to the drawings. In the description and drawings, specific implementations of the present application are disclosed in detail, and part of the implementations in which the principles of the embodiments of the present application may be employed are indicated. It should be understood that the present application is not limited to the described implementations. On the contrary, the embodiments of the present application include all modifications, variations, and equivalents which fall within the scope of the appended claims.

In the embodiments of the present application, the terms “first” and “second” etc., are used to distinguish different elements, but do not represent a spatial arrangement or temporal order, etc., of these elements, and these elements should not be limited by these terms. The term “and/or” includes any and all combinations of one or more associated listed terms. The terms “comprise”, “include”, “have”, etc., refer to the presence of described features, elements, components, or assemblies, but do not exclude the presence or addition of one or more other features, elements, components, or assemblies.

In the embodiments of the present application, the singular forms “a” and “the”, etc., include plural forms, and should be broadly construed as “a type of” or “a class of” rather than being limited to the meaning of “one”. Furthermore, the term “the” should be construed as including both the singular and plural forms, unless otherwise specified in the context. In addition, the term “according to” should be construed as “at least in part according to...” and the term “on the basis of” should be construed as “at least in part on the basis of...”, unless otherwise specified in the context.

The features described and/or illustrated for one implementation may be used in one or more other implementations in the same or similar way, be combined with features in other implementations, or replace features in other implementations. The term “include/comprise” when used herein refers to the presence of features, integrated components, steps, or assemblies, but does not preclude the presence or addition of one or more other features, integrated components, steps, or assemblies.

In some embodiments, an ultrasound imaging system may be used to perform scanning and imaging on a body part of a human body or other living entities. However, the present application is not limited thereto, and the ultrasound imaging system may also be used to perform scanning and imaging on a non-living entity. Moreover, the present application is also applicable to other imaging apparatuses having similar structures or functions (for example, apparatuses for performing imaging by applying visible light, X-rays, a magnetic field, or other physical signals to an object being scanned).

The following is a specific description of the embodiments of the present application with reference to the drawings.

Embodiments of the present application provide an ultrasound imaging system.

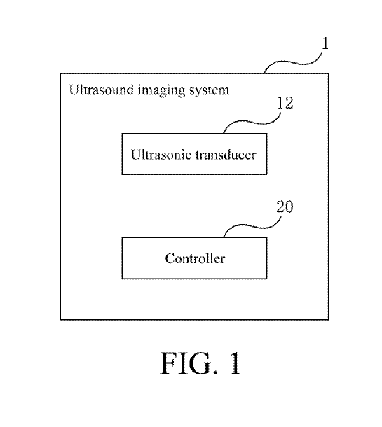

FIG. 1 is a schematic diagram of an ultrasound imaging system according to an embodiment of the present application. As shown in FIG. 1, the ultrasound imaging system 1 includes an ultrasonic transducer 12 and a controller 20. The ultrasonic transducer 12 moves between a first scanning position and a second scanning position to acquire an ultrasound image of an object being scanned. The controller 20 is configured to: detect a region of interest in the ultrasound image in real time during a process in which the ultrasonic transducer 12 moves from the first scanning position to the second scanning position; and dynamically adjust a scanning mode of the ultrasonic transducer 12 according to a detection result of the region of interest, where in different scanning modes, moving speeds of the ultrasonic transducer are different.

According to the above embodiment, the region of interest in the acquired ultrasound image is detected in real time during the process in which the ultrasonic transducer 12 moves from the first scanning position to the second scanning position; and the scanning mode of the ultrasonic transducer is dynamically adjusted according to the detection result of the region of interest, where in different scanning modes, the moving speeds of the ultrasonic transducer are different. Therefore, the moving speed of the ultrasonic transducer 12 can be dynamically adjusted based on the real-time detection result of the region of interest, so that a balance between image quality and scanning time can be achieved, which is conducive to shortening the scanning time of ultrasound imaging and improving the scanning efficiency of the ultrasound imaging system 1 while ensuring the image quality of ultrasound imaging.

In this embodiment of the present application, the process in which the ultrasonic transducer 12 moves from the first scanning position to the second scanning position is referred to as one scanning cycle. By dynamically adjusting the moving speed of the ultrasonic transducer based on the detection result of the region of interest in one scanning cycle, the image quality and scanning efficiency of each scanning cycle can be ensured.

In some embodiments, the ultrasound imaging system 1 may be in various forms. For example, the ultrasound imaging system 1 may be a full-field breast ultrasound imaging system. The structure of the ultrasound imaging system 1 will be exemplarily described below with reference to the accompanying drawings by taking a full-field breast ultrasound imaging system as an example.

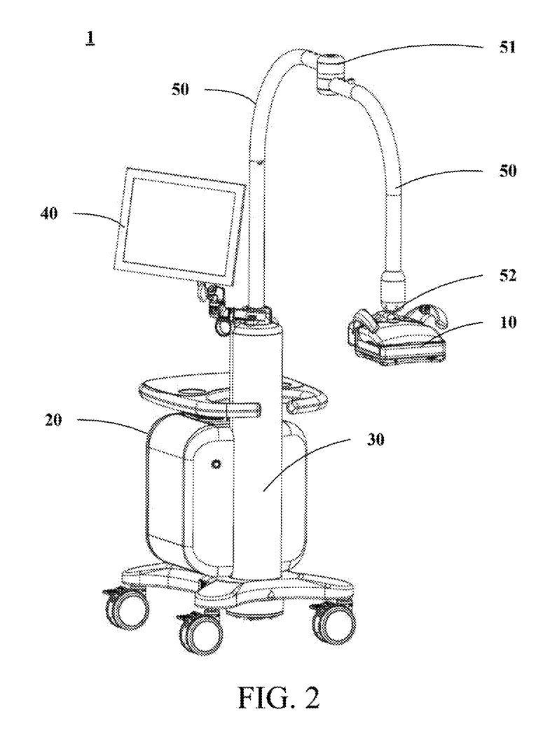

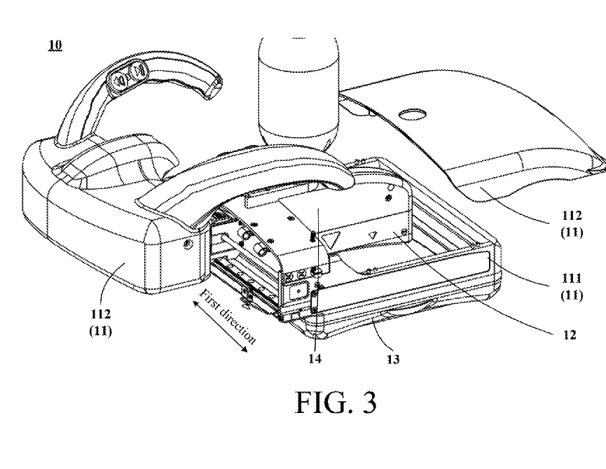

FIG. 2 is a schematic diagram of the structure of an ultrasound imaging system according to an embodiment of the present application. As shown in FIG. 2, the ultrasound imaging system 1 includes an ultrasound imaging assembly 10 and a controller 20. FIG. 3 is a schematic diagram of an ultrasound imaging assembly according to an embodiment of the present application. As shown in FIG. 3, the ultrasound imaging assembly 10 includes a housing 11 and an ultrasonic transducer 12, where the controller 20 may control the ultrasonic transducer 12 to cause the ultrasonic transducer 12 to move in the housing 11. The described first scanning position and second scanning position may be a starting position and an ending position of the ultrasonic transducer 12 moving in the housing 11 in one scanning cycle.

As shown in FIG. 2, in addition to the ultrasound imaging assembly 10 and the controller 20, the ultrasound imaging system 1 may further include a main body frame 30 and a movable support arm 50 including a hinge joint 51 or another similar structure. One end of the support arm 50 is connected to the main body frame 30, and the other end is connected to the ultrasound imaging assembly 10 through a ball-and-socket connector 52 or other similar structures.

In some embodiments, the support arm 50 may be configured to enable the ultrasound imaging assembly 10 to apply a preset pressure to the object being scanned (for example, a breast) during scanning, thereby ensuring that the ultrasound imaging assembly 10 is in close contact with the object being scanned. In this way, ultrasonic signals can be sent and/or received with low attenuation, and relative displacement between the ultrasound imaging assembly 10 and the object being scanned can be avoided, thereby helping to improve imaging quality.

As shown in FIG. 2, the ultrasound imaging system 1 may further include a display 40. The display 40 may be connected to the main body frame 30, and thus the weight of the support arm 50 and the balance mechanism of the support arm 50 would not be affected. Although FIG. 2 shows the display 40 being connected to the main body frame 30, in another example, the display 40 may be connected to another component of the ultrasound imaging system 1.

As shown in FIG. 3, the ultrasound imaging assembly 10 may further include a film assembly 13. The film assembly 13 includes an outer frame and a film. The film is fixedly disposed in the outer frame, and the outer frame is detachably connected to other components (for example, a framework 111 of the housing 11 to be described later) of the ultrasound imaging assembly 10.

During scanning and imaging of the ultrasound imaging system, one surface of the film is at least partially in contact with the ultrasonic transducer 12 of the ultrasound imaging assembly 10, and the other surface of the film is at least partially in contact with the object being scanned. In this way, the ultrasonic transducer 12 can be ensured to send and receive signals with low attenuation, and the object being scanned can be fixed to facilitate scanning. In some embodiments, the film may be a tensioned fabric sheet.

As shown in FIG. 3, the housing 11 includes a framework 111, and the ultrasonic transducer 12 moves within a space enclosed by the framework 111 to cover a preset scanning range. The ultrasonic transducer 12 may move within the framework 111 in various manners. For example, as shown in FIG. 3, the ultrasonic transducer 12 reciprocates in a first direction.

As shown in FIG. 3, the housing 11 may further include an upper housing 112, and the upper housing 112 is disposed on an upper side of the framework 111. The upper side of the upper housing 112 faces the support arm 50.

As shown in FIG. 3, the ultrasound imaging assembly 10 may further include a drive apparatus 14 for driving the ultrasonic transducer 12 to move. The drive apparatus 14 may be disposed on top of the ultrasonic transducer 12, but the present application is not limited thereto. The drive apparatus 14 may alternatively be disposed at other positions of the ultrasonic transducer 12 or connected to the ultrasonic transducer 12 in other manners.

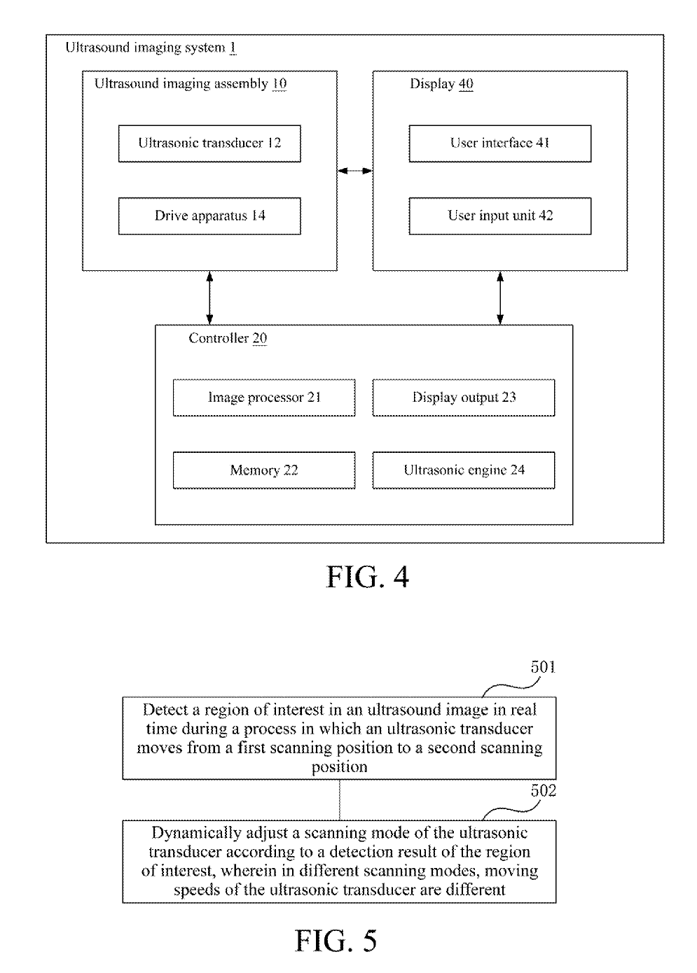

FIG. 4 is a schematic diagram of internal signal interaction of an ultrasound imaging system according to an embodiment of the present application. As shown in FIG. 4, in the ultrasound imaging system 1, there is signal interaction between the ultrasound imaging assembly 10, the controller 20, and the display 40. The ultrasound imaging assembly 10, the controller 20, and the display 40 may be independent components that communicate with each other. However, the present application is not limited thereto, and one or more of these components may also be integrated.

In the ultrasound imaging assembly 10, the ultrasonic transducer 12 may include a transducer array including a plurality of transducer elements. The transducer elements can convert electrical energy into ultrasonic waves for transmission and can detect reflected ultrasonic echoes. The ultrasound imaging assembly 10 can communicate with the controller 20 to send raw scan data (for example, ultrasonic echo signals) to the controller 20.

The controller 20 may include, for example, an image processor 21, a memory 22, display output 23, and an ultrasonic engine 24. The memory 22 can store various types of information. In addition, the memory 22 can further store a control program, and the program can be executed under the control of the image processor 21 or the ultrasonic engine 24.

The image processor 21 can process the received raw scan data to form a displayable image of a tissue sample, for example, to form an image sequence including a plurality of ultrasound slices. But the present application is not limited thereto. The raw scan data may alternatively be processed by a remote processor or the like.

The ultrasonic engine 24 can activate the transducer elements of the ultrasonic transducer 12. In some embodiments, the ultrasonic engine 24 may further activate the drive apparatus 14 in the ultrasound imaging assembly. In addition, the ultrasonic engine 24 may also integrate related functions of the image processor 21.

The controller 20 can communicate with the display 40 via the display output 23, and transmit image data of the displayable image from the ultrasonic engine 24 or the image processor 21 to the display 40. The display 40 may include a user interface 41 configured to display images or other information to an operator. The display 40 may further include a user input unit 42 for receiving operator input. In one example, the user input unit 42 may be a touchscreen of the display 40. However, other types of user input mechanisms are also possible, such as a mouse or a keyboard.

The structure and internal signal interaction of the ultrasound imaging system 1 are exemplarily described above by taking a full-field breast ultrasound imaging system as an example. However, the present application is not limited thereto, and the ultrasound imaging system 1 may also be other types of ultrasound imaging systems.

The actions of the controller 20 in this embodiment of the present application will be exemplarily described below.

FIG. 5 is a schematic diagram of a control method executed by a controller according to an embodiment of the present application. As shown in FIG. 5, the control method includes:

Step 501: Detecting a region of interest in an ultrasound image in real time during a process in which the ultrasonic transducer moves from a first scanning position to a second scanning position; and

Step 502: Dynamically adjusting a scanning mode of the ultrasonic transducer according to a detection result of the region of interest, wherein in different scanning modes, moving speeds of the ultrasonic transducer are different.

In some embodiments, in different scanning modes, imaging parameters of the ultrasound image may also be different. The imaging parameter of the ultrasound image includes at least one of the following parameters: a quantity of compound angles, a quantity of transmit waveforms, and a frame interval.

Therefore, not only can the moving speed of the ultrasonic transducer be dynamically adjusted based on the detection result of the region of interest, but also the image parameter of the ultrasound image can be dynamically adjusted based on the detection result of the region of interest. This can achieve a balance between image quality and image data volume, which helps reduce the overall data volume of an ultrasound image sequence while ensuring the image quality of ultrasound imaging of the region of interest, thereby helping improve the efficiency of a series of subsequent image processing.

In some embodiments, the scanning mode of the ultrasonic transducer may include a first scanning mode (which may also be referred to as a fast scanning mode or the like) and a second scanning mode (which may also be referred to as a slow scanning mode, a local scanning mode, or the like).

In some embodiments, in the first scanning mode, the ultrasonic transducer moves at a first moving speed, and in the second scanning mode, the ultrasonic transducer moves at a second moving speed. The first moving speed is greater than the second moving speed.

For example, in the first scanning mode, the first moving speed may be 21 mm/s, and in the second scanning mode, the second moving speed may be 7 mm/s or a speed lower than 7 mm/s. The present application is not limited thereto, and the first moving speed and the second moving speed may alternatively be other values.

In some embodiments, in the first scanning mode, the first ultrasound image is imaged according to a first imaging parameter, the first imaging parameter including at least one of the following parameters: a first quantity of compound angles, a first quantity of transmit waveforms, and a first frame interval; and in the second scanning mode, the second ultrasound image is imaged according to a second imaging parameter, the second imaging parameter including at least one of the following parameters: a second quantity of compound angles, a second quantity of transmit waveforms, and a second frame interval.

The first quantity of compound angles in the first scanning mode may be less than the second quantity of compound angles in the second scanning mode.

For example, taking an automated breast ultrasound system (ABUS) as an example, ABUS, as a type of full-field breast ultrasound imaging system, is based on plane wave imaging. A larger quantity of compound angles enables better contrast and a better signal-to-noise ratio to be obtained, and helps improve the image quality of the ultrasound image.

The first quantity of transmit waveforms in the first scanning mode may be less than the second quantity of transmit waveforms in the second scanning mode.

For example, in the second scanning mode, three waveforms may be used, for example, one fundamental waveform and two inverted harmonic waveforms, and in the first scanning mode, only the fundamental waveform or an inverse harmonic waveform may be used.

In the second scanning mode, the ultrasound image may be generated by weighted mixing of received signals of the three waveforms. For example, for a near field, a received signal of each harmonic waveform may be given a higher weight, and for a far field, a received signal of the fundamental wave may be given a higher weight. Therefore, penetrability, contrast resolution, and lateral resolution of the ultrasound image can be better balanced, so that the image quality of the ultrasound image can be further improved. The present application is not limited thereto, and the received signals of the three waveforms may alternatively be processed in another manner.

The first frame interval in the first scanning mode may be greater than the second frame interval in the second scanning mode. Because a smaller frame interval can provide better spatial resolution, the image quality of the ultrasound image can be further improved.

In some embodiments, the second scanning mode may be one mode, that is, the second scanning mode may correspond to a set of parameters, and the set of parameters includes the moving speed of the ultrasonic transducer. Alternatively, the set of parameters may further include the imaging parameter of the ultrasound image, for example, at least one of the quantity of compound angles, the quantity of transmit waveforms, and the frame interval.

In some embodiments, the second scanning mode may alternatively include a plurality of sub-modes, and in different sub-modes, moving speeds of the ultrasonic transducer and/or imaging parameters of the ultrasound image are different. For example, the second scanning mode corresponds to a plurality of sets of parameters, and at least one of the moving speed, the quantity of compound angles, the quantity of transmit waveforms, and the frame interval in different sets of parameters is different.

When the second scanning mode includes a plurality of sub-modes, a sub-mode in the second scanning mode may be selected according to feature information of the region of interest when the region of interest is detected, and the ultrasonic transducer is caused to acquire a second ultrasound image of the object being scanned in the selected sub-mode.

In some embodiments, the feature information of the region of interest may include various feature information related to a lesion, for example, the shape, the size, an edge feature, and an internal echo feature of the lesion. The feature information may be used to evaluate the grade of the lesion. The grade of the lesion may be used to represent the severity of the lesion, the nature of the lesion, or the like. For example, the higher the grade of the lesion, the more severe the lesion or the greater the possibility that the lesion is malignant.

For example, if the lesion grade determined based on the feature information of the region of interest is higher, a sub-mode having a slower moving speed and/or a larger quantity of compound angles and/or a larger quantity of transmit waveforms and/or a smaller frame interval is selected from the second scanning mode. Therefore, the moving speed and/or the imaging parameter of the ultrasonic transducer can be selected corresponding to the lesion grade, so that an ultrasound image having image quality matching the lesion grade can be acquired.

In some embodiments, a correspondence between the lesion grade and the sub-mode may be preset, thereby helping improve control efficiency and further shorten the scanning time. One lesion grade may correspond to one sub-mode, or one lesion grade may correspond to a plurality of sub-modes, or a plurality of lesion grades may correspond to one sub-mode, which is not specifically limited in the present application.

The control method of the present application is exemplarily described below by taking the scanning mode including the first scanning mode and the second scanning mode as an example.

In some embodiments, the first scanning mode may be set as a default scanning mode or an initial scanning mode. For example, in step 501, when scanning is started, the ultrasonic transducer may be caused to acquire a first ultrasound image of the object being scanned in the first scanning mode, and a region of interest is detected in real time based on the first ultrasound image.

In some embodiments, in step 501, the region of interest in the ultrasound image may be detected in real time in various manners. For example, a computer-aided diagnosis (CAD) tool may be used to analyze the ultrasound image and perform real-time detection.

For example, during the movement of the ultrasonic transducer, a plurality of first ultrasound images may be acquired at different positions in a moving direction according to a preset first frame interval to form an image sequence. The CAD tool detects the region of interest in real time for each first ultrasound image. The CAD tool may perform detection based on a current first ultrasound image, or may perform detection based on the current first ultrasound image and several first ultrasound images before the current first ultrasound image, which is not specifically limited in the present application.

In some embodiments, in step 502, the first scanning mode and the second scanning mode are dynamically switched according to a detection result of the region of interest, so that in one scanning cycle, at a position corresponding to the region of interest, a second ultrasound image is acquired at least in the second scanning mode, and in another position, a first ultrasound image is acquired in the first scanning mode.

In some embodiments, the scanning mode of the ultrasonic transducer may be switched in various manners.

For example, the ultrasonic transducer acquires a first ultrasound image in the first scanning mode, and when the region of interest is detected, the ultrasonic transducer is caused to acquire a second ultrasound image of the object being scanned in the second scanning mode; the region of interest is detected in real time based on the second ultrasound image; and in response to detecting the disappearance of the region of interest, the ultrasonic transducer is caused to acquire a first ultrasound image of the object being scanned in the first scanning mode. The ultrasonic transducer repeats the above operations until reaching the second scanning position.

Therefore, in one scanning cycle, at the position corresponding to the region of interest, a second ultrasound image is acquired in the second scanning mode, and in another position, a first ultrasound image is acquired in the first scanning mode.

For another example, the ultrasonic transducer acquires a first ultrasound image in the first scanning mode, and when the region of interest is detected, a current scanning position of the ultrasonic transducer is used as a first marked position, and the ultrasonic transducer is caused to continue to acquire the first ultrasound image of the object being scanned in the first scanning mode, to continuously track the region of interest; and in response to detecting the disappearance of the region of interest, a current scanning position of the ultrasonic transducer is used as a second marked position, and the ultrasonic transducer is caused to acquire a second ultrasound image of the object being scanned in the second scanning mode at least between the first marked position and the second marked position; and after the ultrasonic transducer completes scanning in the second scanning mode, the ultrasonic transducer acquires a first ultrasound image of the object being scanned in the first scanning mode. The ultrasonic transducer repeats the above operations until reaching the second scanning position.

Therefore, in one scanning cycle, at the position corresponding to the region of interest, not only a second ultrasound image is acquired in the second scanning mode, but also a first ultrasound image is acquired in the first scanning mode, and in another position, only a first ultrasound image is acquired in the first scanning mode.

A switching manner of the scanning mode of the ultrasonic transducer is described below with reference to specific examples.

### Manner 1:

In some embodiments, when the appearance of the region of interest is detected in the first scanning mode, the ultrasonic transducer is caused to move backward from a current scanning position by a preset distance, and starts to acquire a second ultrasound image of the object being scanned in the second scanning mode.

By causing the ultrasonic transducer to move backward from the current scanning position by the preset distance, it can be ensured that the entire region of interest (for example, a lesion) can be scanned in the second scanning mode, thereby ensuring that each position of the region of interest can be presented in the ultrasound image with higher image quality.

“Appearance of the region of interest” means that the region of interest was not detected based on a previous first ultrasound image, and the region of interest is detected based on a current first ultrasound image.

In some embodiments, the preset distance may be a distance corresponding to a preset quantity of ultrasound images. For example, an ultrasound image in which a lesion is detected for the first time is denoted as slice A, where slice A is the m^{th} slice in an acquired slice sequence. Slice B is the n^{th} slice, and slice B is acquired prior to slice A, that is, n is less than m. The preset distance is a distance corresponding to slice A and slice B. That is, the ultrasonic transducer acquires slice A at the current scanning position, and after moving backward by the preset distance, the ultrasonic transducer returns to a position at which slice B was acquired.

Alternatively, the preset distance may have another meaning. For example, the preset distance may be a preset fixed distance (which may also be referred to as a safety margin). Alternatively, the preset distance may be the sum of the distance between the current scanning position at which slice A is acquired and the position at which slice B was acquired and a safety margin, or the like.

In some embodiments, similar to the sub-modes of the second scanning mode, the preset distance may also be determined based on the feature information of the region of interest. For example, the higher the lesion grade determined based on the feature information of the region of interest, the larger the preset distance.

Alternatively, the preset distance may alternatively be determined based on the moving speed of the ultrasonic transducer and/or the imaging parameter of the ultrasound image in the first scanning mode. For example, the higher the first moving speed, and/or the smaller the first quantity of compound angles, and/or the smaller the first quantity of transmit waveforms, and/or the larger the first frame interval, the larger the preset distance.

In some embodiments, when the disappearance of the region of interest is detected in the second scanning mode, the ultrasonic transducer may be caused to acquire, starting from a current scanning position, a first ultrasound image of the object being scanned in the first scanning mode.

That is, in response to detecting the disappearance of the region of interest, the ultrasonic transducer may be caused to acquire, starting from the current scanning position at which the disappearance of the region of interest was detected in the second scanning mode, a first ultrasound image of the object being scanned in the first scanning mode.

Because the image quality of the second ultrasound image acquired in the second scanning mode is better than the image quality of the first ultrasound image acquired in the first scanning mode, the ultrasonic transducer does not need to move backward by the preset distance when the disappearance of the region of interest is detected.

“Disappearance of the region of interest” means that the region of interest was detected based on a previous second ultrasound image, and the region of interest is not detected based on a current second ultrasound image.

The present application is not limited thereto, and in response to detecting the disappearance of the region of interest, the ultrasonic transducer may be caused to continue to move in the second scanning mode by a first distance, that is, within the first distance, the ultrasonic transducer still acquires a second ultrasound image of the object being scanned in the second scanning mode. After the ultrasonic transducer continues to move by the first distance, the scanning mode of the ultrasonic transducer is switched from the second scanning mode back to the first scanning mode. Therefore, it can be further ensured that each position of the region of interest can be presented in the ultrasound image with higher image quality.

The first distance may be a distance that is the same as the preset distance. The present application is not limited thereto, and the first distance may alternatively be a distance smaller than the aforementioned preset distance. Because the image quality of the second ultrasound image is better than the image quality of the first ultrasound image, a second detection result (that is, the disappearance of the region of interest) based on the second ultrasound image is more reliable than the first detection result (that is, the appearance of the region of interest) based on the first ultrasound image, and correspondingly, the first distance corresponding to the second detection result may be set to be smaller than the preset distance corresponding to the first detection result.

Manner 1 is exemplarily described below with reference to the accompanying drawings.

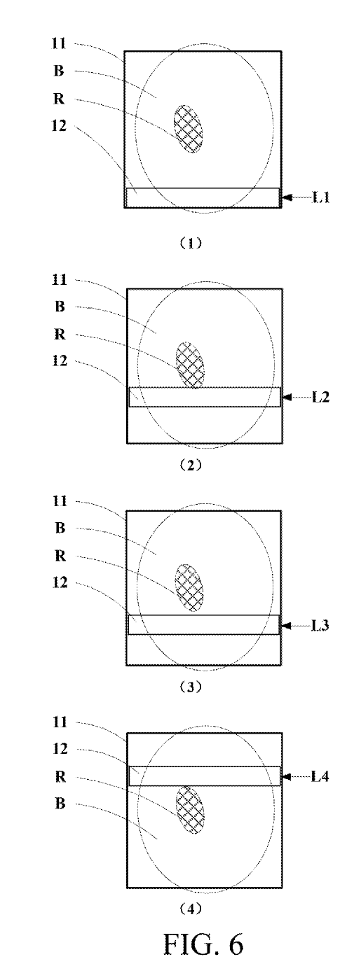

FIG. 6 is a schematic diagram of positions of an ultrasonic transducer according to an embodiment of the present application. As shown in FIG. 6, the ultrasonic transducer 12 moves in the housing 11, the object being scanned B is, for example, a breast, and the region of interest R is, for example, a lesion in the breast.

As shown in (1) of FIG. 6, the ultrasonic transducer 12 acquires, starting from position L1, a first ultrasound image of the object being scanned B in the first scanning mode, and the controller detects the region of interest in real time based on the first ultrasound image.

As shown in (2) of FIG. 6, when the ultrasonic transducer 12 moves to position L2, the controller detects the appearance of the region of interest according to a first ultrasound image at position L2.

As shown in (3) of FIG. 6, the controller causes the ultrasonic transducer 12 to move backward by the preset distance and return to position L3 that has been passed previously, the ultrasonic transducer 12 acquires, starting from position L3, a second ultrasound image of the object being scanned B in the second scanning mode, and the controller detects the region of interest in real time according to the second ultrasound image.

As shown in (4) of FIG. 6, when the ultrasonic transducer 12 moves to position L4, the controller detects the disappearance of the region of interest according to a second ultrasound image at position L4, and the ultrasonic transducer 12 acquires, starting from position L4, a first ultrasound image of the object being scanned B in the first scanning mode.

### Manner 2:

In some embodiments, when the appearance of the region of interest is detected in the first scanning mode, the ultrasonic transducer is caused to acquire, starting from a current scanning position, a second ultrasound image of the object being scanned in the second scanning mode. The ultrasonic transducer emits an ultrasonic signal at a first angle in the first scanning mode, the ultrasonic transducer emits an ultrasonic signal at a second angle in the second scanning mode, and the first angle is different from the second angle.

Therefore, even if the ultrasonic transducer does not move backward from the current scanning position by the preset distance, it can be ensured that the entire region of interest (for example, the lesion) can be scanned in the second scanning mode, thereby ensuring that each position of the region of interest can be presented in the ultrasound image with higher image quality.

For example, in the first scanning mode, the ultrasonic transducer emits ultrasonic waves at the first angle, to acquire a first ultrasound image at a position further ahead of an actual scanning position of the ultrasonic transducer. Upon detection of the appearance of the region of interest, the ultrasonic transducer is switched to the second scanning mode, and the ultrasonic waves are emitted at the second angle starting from the current scanning position of the ultrasonic transducer, to acquire a second ultrasound image aligned with or further behind the actual scanning position of the ultrasonic transducer.

The present application is not limited thereto, and the first angle and the second angle are not limited to the above manners. For example, a position of an ultrasound image acquired at the first angle is aligned with the actual scanning position of the ultrasonic transducer, a position of an ultrasound image acquired at the second angle is behind the actual scanning position of the ultrasonic transducer, and the like.

In some embodiments, when the disappearance of the region of interest is detected in the second scanning mode, the ultrasonic transducer may be caused to acquire, after moving backward from the current scanning position by a second distance, a first ultrasound image of the object being scanned in the first scanning mode.

That is, in response to detecting the disappearance of the region of interest, the ultrasonic transducer may be caused to move backward from the current scanning position at which the disappearance of the region of interest was detected in the second scanning mode, and after moving backward by the second distance, acquire a first ultrasound image of the object being scanned in the first scanning mode.

Because the first angle in the first scanning mode is different from the second angle in the second scanning mode, by causing the ultrasonic transducer to move backward by the second distance, the integrity of the ultrasound image sequence can be ensured.

The second distance may be a distance determined based on the first angle and the second angle.

The present application is not limited thereto, and in response to detecting the disappearance of the region of interest, the ultrasonic transducer may be caused to continue to move in the second scanning mode by a third distance, that is, within the third distance, the ultrasonic transducer still acquires the second ultrasound image of the object being scanned in the second scanning mode. After the ultrasonic transducer continues to move by the third distance, the ultrasonic transducer is caused to move backward from the current scanning position by the second distance, and the scanning mode of the ultrasonic transducer is switched from the second scanning mode back to the first scanning mode. Therefore, it can be further ensured that each position of the region of interest can be presented in the ultrasound image with higher image quality.

Similar to the aforementioned first distance, the third distance may be a distance that is the same as the aforementioned preset distance, or the third distance may be a distance smaller than the aforementioned preset distance.

Manner 2 is exemplarily described below with reference to the accompanying drawings.

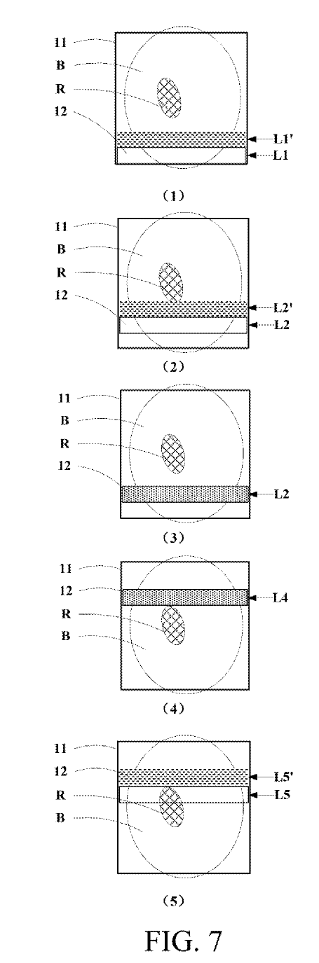

FIG. 7 is another schematic diagram of positions of an ultrasonic transducer according to an embodiment of the present application. As shown in FIG. 7, the ultrasonic transducer 12 moves in the housing 11, the object being scanned B is, for example, a breast, and the region of interest R is, for example, a lesion in the breast.

As shown in (1) of FIG. 7, the ultrasonic transducer 12 acquires, starting from position L1, a first ultrasound image of the object being scanned B in the first scanning mode, where the ultrasonic transducer 12 transmits ultrasonic waves at the first angle to acquire a first ultrasound image at position L1' located in front of position L1. The controller detects the region of interest in real time according to the first ultrasound image.

As shown in (2) of FIG. 7, when the ultrasonic transducer 12 moves to position L2, the controller detects the appearance of the region of interest according to a first ultrasound image at position L2'.

As shown in (3) of FIG. 7, the controller causes the ultrasonic transducer 12 to acquire, starting from position L2, a second ultrasound image of the object being scanned B in the second scanning mode, where the ultrasonic transducer 12 transmits ultrasonic waves at the second angle, to acquire a second ultrasound image at a position aligned with position L2, and the controller detects the region of interest in real time according to the second ultrasound image.

As shown in (4) of FIG. 7, when the ultrasonic transducer 12 moves to position L4, the controller detects the disappearance of the region of interest according to a second ultrasound image at position L4, and the controller causes the ultrasonic transducer 12 to move backward from position L4 to position L5 that has been passed previously and acquire, starting from position L5, a first ultrasound image of the object being scanned B in the first scanning mode, where the ultrasonic transducer 12 transmits ultrasonic waves at the first angle, to acquire a first ultrasound image at position L5' located in front of position L5. It may be understood that, in another embodiment, the backward moving operation may not be performed. A setting manner of the first angle and the second angle can ensure that complete information of the region of interest can be obtained even without moving backward.

### Manner 3:

In some embodiments, after the appearance of the region of interest is detected in the first scanning mode, the scanning mode is not switched immediately, instead, scanning continues to be performed in the first scanning mode, and after the disappearance of the region of interest is detected in the first scanning mode, the scanning mode is switched from the first scanning mode to the second scanning mode, and scanning is performed in the second scanning mode at least between the first marked position and the second marked position corresponding to the region of interest.

Therefore, in one scanning cycle in which the ultrasonic transducer moves from the first scanning position to the second scanning position, a complete image sequence of first ultrasound images can be generated, that is, there is a corresponding first ultrasound image at each position between the first scanning position and the second scanning position. In addition, between the first marked position and the second marked position corresponding to the region of interest, in addition to the first ultrasound images, a second ultrasound image with better image quality is also generated. This can provide more choices for a user to facilitate diagnosis. In addition, because the complete image sequence of first ultrasound images is generated, improvement of the efficiency of a series of subsequent image processing is facilitated.

In some embodiments, Manner 3 may be combined with Manner 1. For example, a third marked position may be calculated based on the first marked position, where the third marked position is moved backward by a preset distance compared with the first marked position. The ultrasonic transducer is caused to acquire a second ultrasound image of the object being scanned in the second scanning mode between the third marked position and the second marked position. Therefore, it can be ensured that the entire region of interest can be scanned in the second scanning mode.

For another example, a fourth marked position may be calculated based on the second marked position, where the fourth marked position is moved forward by a preset distance compared with the second marked position. The ultrasonic transducer is caused to acquire a second ultrasound image of the object being scanned in the second scanning mode between the first marked position and the fourth marked position or between the third marked position and the fourth marked position. Therefore, it can be ensured that the entire region of interest can be scanned in the second scanning mode.

A combination of Manner 3 and Manner 1 is exemplarily described with reference to the accompanying drawings.

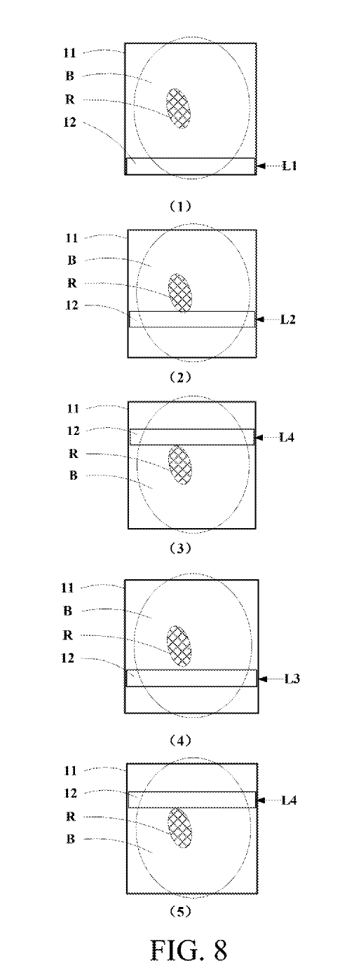

FIG. 8 is another schematic diagram of positions of an ultrasonic transducer according to an embodiment of the present application. As shown in FIG. 8, the ultrasonic transducer 12 moves in the housing 11, the object being scanned B is, for example, a breast, and the region of interest R is, for example, a lesion in the breast.

As shown in (1) of FIG. 8, the ultrasonic transducer 12 acquires, starting from position L1, a first ultrasound image of the object being scanned B in the first scanning mode, and the controller detects the region of interest in real time according to the first ultrasound image.

As shown in (2) of FIG. 8, when the ultrasonic transducer 12 moves to position L2, the controller detects the appearance of the region of interest according to a first ultrasound image at position L2.

As shown in (3) of FIG. 8, the controller causes the ultrasonic transducer 12 to continue to perform scanning in the first scanning mode, and tracks the region of interest in real time according to the first ultrasound image.

As shown in (4) of FIG. 8, when the ultrasonic transducer 12 moves to position L4, the controller detects the disappearance of the region of interest according to a first ultrasound image at position L4, and causes the ultrasonic transducer 12 to move backward to position L3. Position L3 is a position that is moved backward by a preset distance from position L2 at which the appearance of the region of interest was detected. The ultrasonic transducer 12 acquires, starting from position L3, a second ultrasound image of the object being scanned B in the second scanning mode.

As shown in (5) of FIG. 8, when the ultrasonic transducer 12 moves to position L4 again, the controller causes the ultrasonic transducer 12 to continue to acquire, starting from position L4, a first ultrasound image of the object being scanned B in the first scanning mode.

In addition, Manner 3 may alternatively be combined with Manner 2.

For example, the position of the first ultrasound image corresponding to the first angle is ahead of the actual scanning position of the ultrasonic transducer, and the position of the second ultrasound image corresponding to the second angle is aligned with the actual scanning position of the ultrasonic transducer, and a fifth marked position may be calculated based on the second marked position, where the fifth marked position is located in front of the second marked position and is a second distance from the second marked position. Between the first marked position and the fifth marker, a second ultrasound image is acquired in the second scanning mode. After the ultrasonic transducer completes scanning in the second scanning mode, the ultrasonic transducer continues to acquire, starting from the second marked position, a first ultrasound image of the object being scanned in the first scanning mode.

Because the position of the first ultrasound image corresponding to the first angle is ahead of the actual scanning position of the ultrasonic transducer, and the position of the second ultrasound image corresponding to the second angle is aligned with the actual scanning position of the ultrasonic transducer, by causing the ultrasonic transducer to acquire a the second ultrasound image in the second scanning mode between the first marked position and the fifth marked position that is further ahead of the second marked position, it can be ensured that the entire region of interest can be scanned in the second scanning mode.

The switching manner of the scanning mode of the present application is exemplarily described above with reference to the accompanying drawings. The present application is not limited thereto, and the scanning mode may alternatively be switched in another manner.

In some embodiments, the first ultrasound image and the second ultrasound image acquired in Manner 1 or Manner 2 may be stitched together in various manners to generate the ultrasound image sequence. For example, an overlapping portion of the first ultrasound image and the second ultrasound image is determined, the overlapping portion in the first ultrasound image is deleted, and the overlapping portion in the second ultrasound image is retained. Thus, a complete sequence of ultrasound images which are in a one-to-one correspondence with positions between the first scanning position and the second scanning position can be generated.

The overlapping portion of the first ultrasound image and the second ultrasound image may be determined in various manners. For example, the overlapping portion of the first ultrasound image and the second ultrasound image may be determined based on a position of the ultrasonic transducer during scanning mode switching, or may be directly determined according to the first ultrasound image and the second ultrasound image, or may be determined based on the position of the ultrasonic transducer during scanning mode switching and the first ultrasound image and the second ultrasound image themselves, which is not specifically limited in the present application.

In some embodiments, for the first ultrasound image and the second ultrasound image acquired in Manner 1 or Manner 2, first position information of the second ultrasound image may be recorded, and the first position information is used to indicate a range of the second ultrasound image in the ultrasound image sequence.

In some embodiments, for the first ultrasound image and the second ultrasound image acquired in Manner 3, second position information of the second ultrasound image overlapping with the first ultrasound image may be recorded. The second position information is used to indicate a range of the second ultrasound image in the first ultrasound image. For example, the overlapping portion of the first ultrasound image and the second ultrasound image may be determined in the foregoing manner, and the second position information may be determined according to position information of the overlapping portion.

In some embodiments, marking information for indicating the range of the second ultrasound image may be displayed by means of a display. Therefore, it is convenient for the user to clearly understand which regions in a displayed image correspond to ultrasound images with better image quality, so that the user focuses on that portion of the ultrasound images, which helps improve the accuracy and efficiency of diagnosis.

The display herein may be the display 40 of the ultrasound imaging system 1 shown in FIG. 2. However, the present application is not limited thereto, and the display herein may alternatively be a display of another device.

The marking information may be determined according to the first position information or the second position information. The marking information may be displayed in various manners. For example, the range of the second ultrasound image may be directly marked in the displayed image. The displayed image may be a reconstructed ultrasound image generated by means of image reconstruction. The present application is not limited thereto, and the marking information may alternatively be displayed in another manner, for example, the position at which the second ultrasound image is acquired is shown on an axis representing the scanning position.

In some embodiments, image reconstruction may be performed based on the first ultrasound image and the second ultrasound image, to generate a reconstructed ultrasound image and cause the display to display the reconstructed ultrasound image. The marking information is used to indicate a portion of the reconstructed ultrasound image corresponding to the second ultrasound image.

Because the image quality of the first ultrasound image is different from the image quality of the second ultrasound image, the reconstructed ultrasound image generated based on the first ultrasound image and the second ultrasound image may exhibit phenomena of difference in resolution, contrast, and the like near positions adjacent to the first ultrasound image and the second ultrasound image, and by displaying the marking information in the reconstructed ultrasound image, the user can be prevented from being confused about the above phenomena, which helps improve the confidence of the user in making a diagnosis based on the reconstructed ultrasound image.

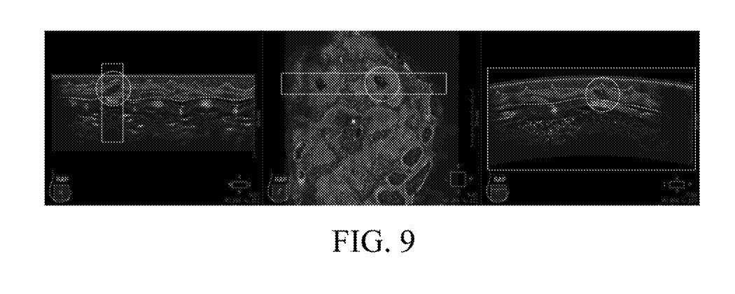

FIG. 9 is a schematic diagram of reconstructed ultrasound images and marking information according to an embodiment of the present application. As shown in FIG. 9, reconstructed ultrasound images at a plurality of angles may be displayed by means of the display, and in the reconstructed ultrasound images at the plurality of angles, the position of the region of interest and the marking information used to indicate the range of the second ultrasound image are respectively shown in different forms.

As shown in FIG. 9, the display sequentially displays the reconstructed ultrasound images of sagittal, coronal, and transverse orientations, where in each image, the range of the region of interest is indicated by a circle, and the range of the second ultrasound image is indicated by a rectangle.

In some embodiments, when the complete image sequence of first ultrasound images is acquired, image reconstruction may be performed based on the first ultrasound images, to generate a reconstructed ultrasound image, and cause the display to display the reconstructed ultrasound image. The marking information is used to indicate a range in the reconstructed ultrasound image that corresponds to the second ultrasound image. In response to the marking information being operated upon, the display may display the second ultrasound image corresponding to the marking information.

Because the first ultrasound image has a smaller data volume than the second ultrasound image, performing image reconstruction based on the first ultrasound image can improve the efficiency of image processing. In addition, by displaying the marking information in the reconstructed ultrasound image, the user can cause the display to display the second ultrasound image with better image quality by operating upon the marking information (for example, selecting or dragging the marking information, etc.), so that the user can flexibly select, according to usage habits, an ultrasound image that the user wishes to observe.

The foregoing description merely schematically illustrates the embodiments of the present application, but the present application is not limited thereto. For example, the order of execution between operations may be appropriately adjusted. In addition, some other operations may be added or some operations may be omitted. Those skilled in the art can make appropriate variations according to the above content, rather than being limited by the disclosure of the foregoing accompanying drawings.

The foregoing description relates only to the components or modules related to the present application, but the present application is not limited thereto. The ultrasound imaging system may further include other components or modules, or some components or modules may be omitted, and reference may be made to the related art for details of these components or modules.

In addition, for simplicity, the above figures only exemplarily illustrate connection relationships or signal directions between various components or modules, but it should be clear to those skilled in the art that various related technologies such as bus connection may be used. The various components or modules may be implemented by means of hardware facilities such as a processor, a memory, a transmitter and a receiver. The implementation of the present application is not limited thereto.

According to the above embodiment, the region of interest in the acquired ultrasound image is detected in real time during the process in which the ultrasonic transducer moves from the first scanning position to the second scanning position; and the scanning mode of the ultrasonic transducer is dynamically adjusted according to the detection result of the region of interest, where in different scanning modes, the moving speeds of the ultrasonic transducer are different. Therefore, the moving speed of the ultrasonic transducer can be dynamically adjusted based on the real-time detection result of the region of interest, so that a balance between image quality and scanning time can be achieved, which is conducive to shortening the scanning time of ultrasound imaging and improving the scanning efficiency of the ultrasound imaging system while ensuring the image quality of ultrasound imaging.

An embodiment of the present application further provides an imaging method for an ultrasound imaging system. The ultrasound imaging system includes an ultrasonic transducer. The imaging method includes: causing the ultrasonic transducer to move between a first scanning position and a second scanning position to acquire an ultrasound image of an object being scanned, where a region of interest in the ultrasound image is detected in real time during a process in which the ultrasonic transducer moves from the first scanning position to the second scanning position, a scanning mode of the ultrasonic transducer is dynamically adjusted according to a detection result of the region of interest, and in different scanning modes, moving speeds of the ultrasonic transducer are different.

In some embodiments, in different scanning modes, imaging parameters of the ultrasound image are different, where the imaging parameter includes at least one of the following parameters: a quantity of compound angles, a quantity of transmit waveforms, and a frame interval.

The ultrasound imaging system described in the embodiment of the present application can correspond to the ultrasound imaging system 1 described in the foregoing embodiments, and the imaging method described in the embodiment of the present application can correspond to the method described in the foregoing embodiments. For the specific content, reference may be made to the description in the foregoing embodiments.

An embodiment of the present application further provides a computer-readable program, where the program, when executed, causes a computer to perform, in an ultrasound imaging system, the method described in the foregoing embodiments.

An embodiment of the present application further provides a storage medium storing a computer-readable program, where the computer-readable program causes a computer to perform, in an ultrasound imaging system, the method described in the foregoing embodiments.

An embodiment of the present application further provides a computer program product at least including a computer program, where the computer program, when executed by a processor, causes an ultrasound imaging system to perform the control method described in the foregoing embodiments.

The foregoing system, apparatus, and method of the present application can be implemented by hardware or by hardware in combination with software. The present application relates to such a computer-readable program that when executed by a logic component, the program causes the logic component to implement the foregoing apparatus or a constituent component, or causes the logic component to implement various methods or steps as described above. The present application further relates to a storage medium for storing the above program, such as a hard disk, a disk, an optical disk, a DVD, a flash memory, etc.

The method/apparatus described in view of the embodiments of the present application may be directly embodied as hardware, a software module executed by a processor, or a combination of the two. For example, one or more of the functional block diagrams and/or one or more combinations of the functional block diagrams shown in the drawings may correspond to either respective software modules or respective hardware modules of a computer program flow. The foregoing software modules may respectively correspond to the steps shown in the figures. The foregoing hardware modules can be implemented, for example, by firming the software modules using a field-programmable gate array (FPGA).

The software modules may be located in a RAM, a flash memory, a ROM, an EPROM, an EEPROM, a register, a hard disk, a portable storage disk, a CD-ROM, or any other form of storage medium known in the art. The storage medium may be coupled to a processor, so that the processor can read information from the storage medium and can write information into the storage medium. Alternatively, the storage medium may be a constituent component of the processor. The processor and the storage medium may be located in an ASIC. The software modules may be stored in a memory, and may alternatively be stored in a removable memory card. For example, if the device uses a large-capacity MEGA-SIM card or a large-capacity flash memory apparatus, the software modules can be stored in the MEGA-SIM card or the large-capacity flash memory apparatus.

One or more of the functional blocks and/or one or more combinations of the functional blocks shown in the accompanying drawings may be implemented as a general-purpose processor, a digital signal processor (DSP), an application-specific integrated circuit (ASIC), a field-programmable gate array (FPGA) or other programmable logic devices, discrete gate or transistor logic devices, a discrete hardware assembly, or any appropriate combination thereof for executing the functions described in the present application. The one or more functional blocks and/or the one or more combinations of the functional blocks shown in the accompanying drawings may also be implemented as a combination of computing devices, such as a combination of a DSP and a microprocessor, multiple microprocessors, one or more microprocessors in communication combination with a DSP, or any other such configuration.

The present application is described above with reference to specific implementations. However, it should be clear to those skilled in the art that the foregoing description is merely illustrative and is not intended to limit the scope of protection of the present application. Various variations and modifications may be made by those skilled in the art according to the spirit and principle of the present application, and these variations and modifications also fall within the scope of the present application. For example, each of the above embodiments may be used independently, or one or more of the above embodiments may be combined.

Preferred implementations of the present application are described above with reference to the accompanying drawings. Many features and advantages of the implementations are clear according to the detailed description. Therefore, the appended claims are intended to cover all these features and advantages that fall within the true spirit and scope of these implementations. In addition, as many modifications and changes could be easily conceived of by those skilled in the art, the implementations of the present application are not limited to the illustrated and described precise structures and operations, but can encompass all appropriate modifications, changes, and equivalents that fall within the scope of the implementations.

1. An ultrasound imaging system, comprising:
an ultrasonic transducer, configured to move between a first scanning position and a second scanning position to acquire an ultrasound image of an object being scanned; and
a controller, configured to:
detect a region of interest in the ultrasound image in real time during a process in which the ultrasonic transducer moves from the first scanning position to the second scanning position; and
dynamically adjust a scanning mode of the ultrasonic transducer according to a detection result of the region of interest, wherein in different scanning modes, moving speeds of the ultrasonic transducer are different.

2. The system according to claim 1, wherein
in different scanning modes, imaging parameters of the ultrasound image are different, wherein the imaging parameter comprises at least one of the following parameters: a quantity of compound angles, a quantity of transmit waveforms, and a frame interval.

3. The system according to claim 1, wherein
detecting a region of interest in the ultrasound image in real time comprises:
causing the ultrasonic transducer to acquire a first ultrasound image of the object being scanned in a first scanning mode; and
detecting the region of interest in real time according to the first ultrasound image; and
dynamically adjusting a scanning mode of the ultrasonic transducer according to a detection result of the region of interest comprises:
when the region of interest is detected, causing the ultrasonic transducer to acquire a second ultrasound image of the object being scanned in a second scanning mode.

4. The system according to claim 3, wherein
the second scanning mode comprises a plurality of sub-modes, and in different sub-modes, moving speeds of the ultrasonic transducer and/or imaging parameters of the ultrasound image are different.

5. The system according to claim 4, wherein when the region of interest is detected, causing the ultrasonic transducer to acquire a second ultrasound image of the object being scanned in a second scanning mode comprises:
selecting the sub-mode in the second scanning mode according to feature information of the region of interest when the region of interest is detected; and
causing the ultrasonic transducer to acquire the second ultrasound image of the object being scanned in the selected sub-mode.

6. The system according to claim 3, wherein when the region of interest is detected, causing the ultrasonic transducer to acquire a second ultrasound image of the object being scanned in a second scanning mode comprises:
when the appearance of the region of interest is detected, causing the ultrasonic transducer to move backward from a current scanning position by a preset distance; and
starting to acquire the second ultrasound image of the object being scanned in the second scanning mode.

7. The system according to claim 3, wherein when the region of interest is detected, causing the ultrasonic transducer to acquire a second ultrasound image of the object being scanned in a second scanning mode comprises:
when the appearance of the region of interest is detected, causing the ultrasonic transducer to acquire, starting from a current scanning position, the second ultrasound image of the object being scanned in the second scanning mode,
wherein the ultrasonic transducer emits an ultrasonic signal at a first angle in the first scanning mode, the ultrasonic transducer emits an ultrasonic signal at a second angle in the second scanning mode, and the first angle is different from the second angle.

8. The system according to claim 6, wherein the controller is further configured to:
stitch the first ultrasound image and the second ultrasound image, the stitching comprising:
determining an overlapping portion of the first ultrasound image and the second ultrasound image; and
deleting the overlapping portion in the first ultrasound image, and retaining the overlapping portion in the second ultrasound image.

9. The system according to claim 3, wherein when the region of interest is detected, causing the ultrasonic transducer to acquire a second ultrasound image of the object being scanned in a second scanning mode comprises:
when the appearance of the region of interest is detected, using a current scanning position of the ultrasonic transducer as a first marked position, and causing the ultrasonic transducer to continue to acquire the first ultrasound image of the object being scanned in the first scanning mode, to continuously track the region of interest; and
in response to detecting the disappearance of the region of interest, using the current scanning position of the ultrasonic transducer as a second marked position, and causing the ultrasonic transducer to acquire the second ultrasound image of the object being scanned in the second scanning mode at least between the first marked position and the second marked position.

10. The system according to claim 9, wherein the controller is further configured to:
calculate a third marked position based on the first marked position, the third marked position being moved backward by a preset distance compared with the first marked position; and
cause the ultrasonic transducer to acquire the second ultrasound image of the object being scanned in the second scanning mode between the third marked position and the second marked position.

11. The system according to claim 3, wherein dynamically adjusting a scanning mode of the ultrasonic transducer according to a detection result of the region of interest further comprises:
causing the ultrasonic transducer to acquire the second ultrasound image of the object being scanned in the second scanning mode;
detecting the region of interest in real time according to the second ultrasound image; and
in response to detecting the disappearance of the region of interest, causing the ultrasonic transducer to acquire a first ultrasound image of the object being scanned in the first scanning mode.

12. The system according to claim 11, wherein in response to detecting the disappearance of the region of interest, causing the ultrasonic transducer to acquire a first ultrasound image of the object being scanned in the first scanning mode comprises:
in response to detecting the disappearance of the region of interest, causing the ultrasonic transducer to acquire, starting from a current scanning position, the first ultrasound image of the object being scanned in the first scanning mode.

13. The system according to claim 3, wherein
in the first scanning mode, the ultrasonic transducer moves at a first moving speed; and
in the second scanning mode, the ultrasonic transducer moves at a second moving speed,
wherein the first moving speed is greater than the second moving speed.

14. The system according to claim 3, wherein
in the first scanning mode, the first ultrasound image is imaged according to a first imaging parameter, the first imaging parameter comprising at least one of the following parameters: a first quantity of compound angles, a first quantity of transmit waveforms, and a first frame interval; and
in the second scanning mode, the second ultrasound image is imaged according to a second imaging parameter, the second imaging parameter comprising at least one of the following parameters: a second quantity of compound angles, a second quantity of transmit waveforms, and a second frame interval,
wherein the first quantity of compound angles is less than the second quantity of compound angles, and/or the first quantity of transmit waveforms is less than the second quantity of transmit waveforms, and/or the first frame interval is greater than the second frame interval.

15. The system according to claim 3, further comprising a display, wherein the controller is further configured to cause the display to display marking information indicating a range of the second ultrasound image.

16. The system according to claim 15, wherein the controller is further configured to:
perform image reconstruction based on the first ultrasound image and the second ultrasound image, to generate a reconstructed ultrasound image, and cause the display to display the reconstructed ultrasound image, wherein the marking information is used to indicate a portion of the reconstructed ultrasound image corresponding to the second ultrasound image.

17. The system according to claim 15, wherein the controller is further configured to:
perform image reconstruction based on the first ultrasound image, to generate a reconstructed ultrasound image, and cause the display to display the reconstructed ultrasound image; and
in response to the marking information being operated upon, display the second ultrasound image corresponding to the marking information.

18. An imaging method for an ultrasound imaging system, the ultrasound imaging system comprising an ultrasonic transducer, the method comprising:
controlling the ultrasonic transducer to move between a first scanning position and a second scanning position to acquire an ultrasound image of an object being scanned;
detecting, in real time, a region of interest in the ultrasound image during a process in which the ultrasonic transducer moves from the first scanning position to the second scanning position; and
adjusting a scanning mode of the ultrasonic transducer according to a detection result of the region of interest,
wherein, in different scanning modes, moving speeds of the ultrasonic transducer are different.

19. The method according to claim 18, wherein
in different scanning modes, imaging parameters of the ultrasound image are different, wherein the imaging parameter comprises at least one of the following parameters: a quantity of compound angles, a quantity of transmit waveforms, and a frame interval.
